(19) United States
(12) Patent Application Publication
(10) Pub. No.: US 2024/0197273 A1
(43) Pub. Date: Jun. 20, 2024

(54) ULTRASOUND-GUIDED PREDICTION OF LOCAL BOLUS VELOCITIES

(71) Applicant: KONINKLIJKE PHILIPS N.V., EINDHOVEN (NL)

(72) Inventors: Sven Peter Prevrhal, Hamburg (DE); Seyedali Sadeghi, Melrose, MA (US); Shyam Bharat, Arlington, MA (US); Claudia Errico, Medford, MA (US)

(21) Appl. No.: 18/288,073

(22) PCT Filed: Apr. 21, 2022
(86) PCT No.: PCT/EP2022/060473

§ 371 Date: Oct. 24, 2023

Related U.S. Application Data
(60) Provisional application No. 63/179,619, filed on Apr. 26, 2021.

(51) Int. Cl. A61B 6/50; A61B 6/00; A61B 6/03; A61B 6/04; A61B 8/00; A61B 8/06; A61B 8/08; G16H 50/50

(52) U.S. Cl. CPC A61B 6/507; A61B 6/032; A61B 6/0407; A61B 6/504; A61B 6/5247; A61B 8/06; A61B 8/0891; A61B 8/4416; A61B 8/481; A61B 8/488; G16H 50/50

(57) ABSTRACT

A system (100) for medical imaging is described. The system (100) includes: a computer tomography (CT) device (502) having a table (106); a processor; and a memory (130) that stores instructions. When executed by the processor, the instructions cause the processor to: compile ground truth data from measured BFS at a plurality of locations of an examined portion of a body, including each of a plurality of specific locations (201); train a computational model (211) to predict BFS using the ground truth data; infer BFS's throughout the examined portion of the body based on the computational model (211); and determine a contrast medium injection duration, or a speed of a table (106), or both, prior to a CT scan (402) based on the inferred BFS's. A method (600) and a tangible non-transitory computer readable medium are also disclosed.

### BACKGROUND

Peripheral artery disease (PAD) is a serious ailment that is caused by the undersupply of oxygenated blood to a limb or other portion of the body. For example, PAD can result from a variety of diseases, such as diabetes and renal disease. When PAD is not properly treated in a limb, for example, limb ischemia can occur, resulting in loss of limb, and even death.

Diagnosis of PAD may be carried out via clinical tests such as clinical examination, Ankle Brachial Index (ABI), and via imaging. Computer tomography (CT) angiography (CTA) is the most common imaging technique used to diagnose PAD. In CTA, an intravenous contrast media (CM) is used to achieve arterial enhancement, which is proportional to the CM iodine concentration, and CM flow rate. Beneficially, the CT scan is triggered at peak CM concentration to deliver acceptable image contrast. For longer arterial paths, a “bolus-chasing” technique is used in an effort to center the CT detector on the advancing peak enhancement. As a result, it is beneficial to try to match the speed of the patient table as it enters the gantry to the speed of the blood and the speed of the CM as it flows through the arteries being imaged.

Among other drawbacks of known CT scanning methods and systems, difficulties in determination of blood flow and blood flow speed (BFS) are exacerbated when the artery of interest is located in a peripheral portion of a body, such as in a lower portion of an appendage (below-the-knee, for example). This is due to reduced blood flow caused by upstream arterial stenosis and calcifications. In many cases, the blood flow is reduced to a slow trickle downstream from the stenosis. This makes location of the contrast bolus peak difficult to predict. Additional dependencies to such local vascular pathologies, including occlusion, stenosis and aneurysm, are the distance to venous access point, cardiac output and recruitment status of collateral arteries. Vessel diameters also vary greatly by age, gender and arterial tree level and also change significantly from systole to diastole. This makes choosing the optimal table feed speed for the CT acquisition a significant challenge.

Another challenge in known systems and methods of arterial imaging is selecting the correct injection protocol. Since BFS varies along an artery for reasons alluded to above, the transit of CM bolus in diseased areas is often considerably slower. In known systems and methods, the time window of contrast enhancement often must be widened to properly scan the diseased areas. This may require long (biphasic) contrast injections, with a corresponding iodine burden for the patient. Since iodine is excreted via the kidneys, this is especially troublesome since PAD patients often have renal co-disease. Contrast-induced nephropathy (CIN) is a major problem associated with the excessive use of contrast in PAD diagnosis/treatment.

Furthermore, increased scan durations to properly image a diseased artery consequently increase radiation exposure of the patient. Long-term ill-effects to the patient are a risk, especially when the imaging protocol is as involved as it is with percutaneous transluminal coronary angioplasty (pCTA). Radiation exposure in PAD may be significant because patients may accumulate exposure if referred to percutaneous intervention, which also uses X-ray imaging guidance, and may also be referred to follow-up pCTA. For younger patients, additional dose increases risk of radiation-induced long-term consequences.

What is needed, therefore, is a method and system for imaging arterial blood flow in a patient that overcomes at least the drawbacks of known methods and systems described above.

### SUMMARY

According to an aspect of the present disclosure, a method performing a computer tomography (CT) angiography is described. The method comprises: applying ultrasound (US) to a portion of a body to provide an image of arterial blood flow; measuring BFS in an artery at a plurality of locations of an examined portion of a body, including a plurality of specific locations of the body; compiling ground truth data from the measured BFS at the plurality of locations of an examined portion of a body, including each of the plurality of specific locations; predicting with a computational model a BFS using the ground truth data; inferring BFS's throughout the portion of the body based on the computational model; and determining contrast medium injection duration, or a speed of a table, or both, prior to a CT scan based on the inferred BFS's.

According to another aspect of the present disclosure, a system for medical imaging is described. The system comprises: a computer tomography (CT) device comprising a table; a processor; and a memory that stores instructions. When executed by the processor, the instructions cause the processor to: compile ground truth data from measured BFS at a plurality of locations of an examined portion of a body, including each of a plurality of specific locations; may use a computational model to predict BFS using the ground truth data; infer BFS's throughout the examined portion of the body based on the computational model; and determine a contrast medium injection duration, or a speed of a table, or both, prior to a CT scan based on the inferred BFS's.

According to another aspect of the present disclosure, a tangible, non-transitory computer readable medium that stores instructions is described. When executed by a processor, the instructions cause the processor to: compile ground truth data from measured BFS at a plurality of locations of an examined portion of a body, including each of a plurality of specific locations; predict with a computational model a predicted BFS using the ground truth data; infer BFS's throughout the portion of the body based on the computational model; and determine a contrast medium injection duration, or a speed of a table, or both, prior to a CT scan based on the inferred BFS's.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for imaging a portion of a body, according to a representative embodiment.

FIG. 2 is a simplified flow diagram showing a method and system for imaging blood flow, according to a representative embodiment.

FIG. 3 is a simplified flow diagram showing a method of training a computational model to predict BFS using the ground truth data, according to a representative embodiment.

FIG. 4A is a simplified flow diagram showing a method of inferring BFS's throughout a portion of the body based on the computational model according to a representative embodiment.

FIG. 4B shows contrast media injection levels in limbs useful in understanding the benefits of various aspects of the representative embodiments.

FIG. 5 is a simplified block diagram of a system for conducting an imaging scan, according to a representative embodiment.

FIG. 6 is a simplified flow diagram showing a method of imaging a portion of a body including training a computational model, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

By the present teachings, contrast-enhanced CT angiography is described for use in imaging and diagnosis of PAD and similar conditions. In accordance with various representative embodiments, a computational model is disclosed that has as input arterial BFS's assessed by Doppler or volume flow US exam at indicative anatomical locations, prior to CT angiography. Illustratively, the model is executed right before the CT exam, and corrects for differences in physiology and simulates current and locally resolved peripheral BFS conditions. These are used to determine optimal acquisition parameters such table feed speed or CT helical pitch, and/or well as a rate of contrast media injection. Beneficially, image quality is improved and use of X-ray dose, contrast media, and resulting adverse effects are reduced.

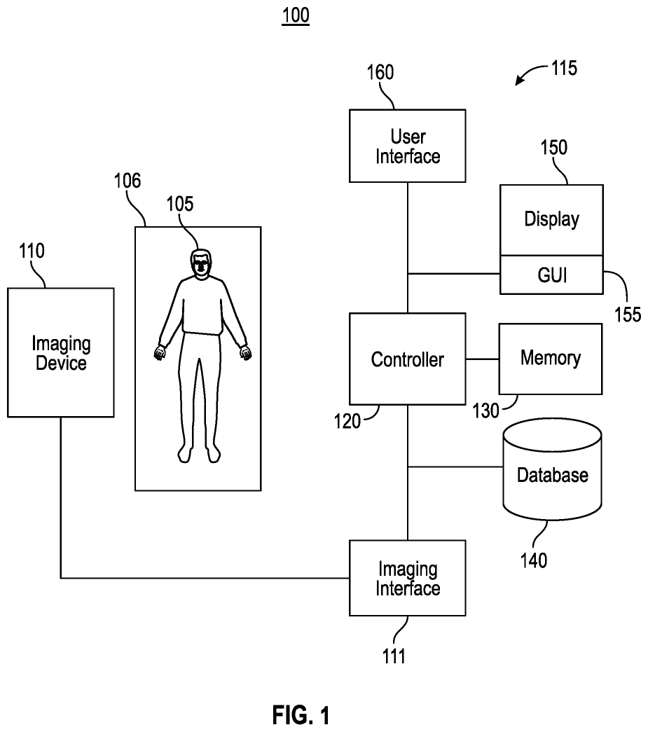

FIG. 1 is a simplified block diagram of a system for imaging a portion of a body, according to a representative embodiment.

Referring to FIG. 1, an imaging system 100 includes an imaging device 110 and a computer system 115 for controlling imaging of a region of interest in a patient 105 on a table 106. The imaging device 110 may be any type of medical imaging device capable of providing an image scan of the region of interest in the patient 105. In accordance with representative embodiments described below, the imaging device is a CT scan imaging device or other imaging modality compatible with the methods and systems of the present teachings described herein. Notably, as will become clearer as the present description continues, US imaging is carried out with a selected US imaging device to construct a computational model described below. Notably, and as will become clearer as the present description continues, the selected US imaging device is not typically a component of the imaging system 100.

The computer system 115 receives image data from the imaging device 110, and stores and processes the imaging data according to the embodiments discussed herein. The computer system 115 includes a controller 120, a memory 130, a database 140 and a display 150.

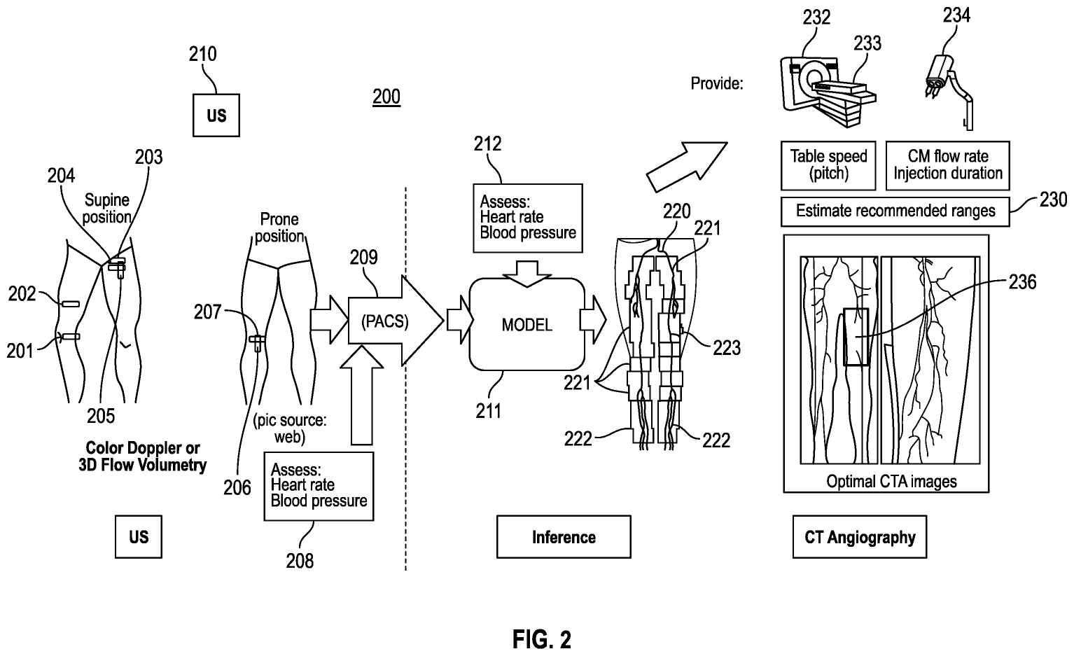

The controller 120 interfaces with the imaging device 110 through an imaging interface 111. The memory 130 stores instructions executable by the controller 120. When executed, and as described more fully below, the instructions cause the controller 120 to implement processes that include inferring BFS's throughout a portion of the body based on a computational model, determining contrast medium injection duration, and determining a speed of the table 106 prior to a CT scan based on the inferred BFS's described below with reference to FIGS. 2-6, for example. In addition, the controller 120 may implement additional operations based on executing instructions, such as instructing or otherwise communicating with another element of the computer system 115, including the database 140 and the display 150, to perform one or more of the above-noted processes.

The controller 120 is representative of one or more processing devices, and is configured to execute software instructions to perform functions as described in the various embodiments herein. The controller 120 may be implemented by field programmable gate arrays (FPGAs), application specific integrated circuits (ASICs), a general purpose computer, a central processing unit, a computer processor, a microprocessor, a microcontroller, a state machine, programmable logic device, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Programs have software instructions performed by one or multiple processors that may be within the same computing device or which may be distributed across multiple computing devices.

The memory 130 may include a main memory and/or a static memory, where such memories may communicate with each other and the controller 120 via one or more buses. The memory 130 stores instructions used to implement some or all aspects of methods and processes described herein. The memory 130 may be implemented by any number, type and combination of random access memory (RAM) and read-only memory (ROM), for example, and may store various types of information, such as software algorithms, which serves as instructions, which when executed by a processor cause the processor to perform various steps and methods according to the present teachings. For example, in accordance with various representative embodiments, the memory 130 that stores instructions, which when executed by the processor, cause the processor to: compile ground truth data (GTD) from measured BFS at a plurality of locations of an examined portion of a body, including each of a plurality of specific locations; train a computational model to predict BFS using the ground truth data; and infer BFS's throughout the portion of the body based on the computational model. The computational model, which is an artificial intelligence (AI) model, may be stored in the memory 130. The computational model may be a known AI model including recurring neural networks (RNNs) and other neural network based models, and computer programs, all of which are executable by the controller 120.

More generally, although not necessarily, the compiling of the GTD from measured BFS at a plurality of locations of an examined portion of a body, including each of a plurality of specific locations, and the training of the computational model to predict BFS using the GTD, may be done using another processor and memory that are not necessarily part of the computer system 115 or the imaging system 100. In this case, after being trained, the computational model may be stored as executable instructions in memory 130, for example, to be executed by a processor of the controller 120. Furthermore, updates to the computational model may also be provided to the computer system 115 and stored in memory 130. Finally, and as will be apparent to one of ordinary skill in the art having the benefit of the present disclosure, according to a representative embodiment, the computational model may be stored in a memory and executed by a processor that is not part of the computer system 115, but rather is connected to the imaging device 115 through an external link (e.g., a known type of internet connection). Just by way of illustration, the computational model may be stored as executable instructions in a memory, and executed by a server that is remote from the imaging device (e.g., a CT scanner). When executed by the processor in the remote server, the instructions cause the processor to compile ground truth data (GTD) from measured BFS at a plurality of locations of an examined portion of a body, including each of a plurality of specific locations; train a computational model to predict BFS using the ground truth data; and infer BFS's throughout the portion of the body based on the computational model. Moreover, the remote server may be configured to determine a contrast medium injection duration, or a speed of a table, or both, prior to an image scan based on the inferred blood flow speeds. The determined contrast medium injection duration, or table feed speed, or both, can then be provided to the imaging device for an image scan to be executed.

The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 130 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 130 may store software instructions and/or computer readable code that enable performance of various functions. The memory 130 may be secure and/or encrypted, or unsecure and/or unencrypted.

Similarly, the database 140 stores data and executable instructions used to implement some or all aspects of methods and processes described herein. As will be described more fully below, the database 140 illustratively stores BFS, contrast medium (CM) injector speed, or table feed speed, or both, for a particular detection (CT) detector width. Notably, the database 140 can be foregone, and all data and executable instructions can be stored in memory 130.

For a set of parameters, a bolus duration of CM, or a table feed speed, or both, for a patient can be provided for a particular CT run. Alternatively or additionally, and again as will be described more fully below, the database may store executable instructions that cause a processor to determine algorithmically the requisite bolus duration, or the table feed speed, or both.

The database 140 may be implemented by any number, type and combination of RAM and ROM, for example, and may store various types of information, such as software algorithms, AI models including RNN and other neural network based models, and computer programs, all of which are executable by the controller 120. The various types of ROM and RAM may include any number, type and combination of computer readable storage media, such as a disk drive, flash memory, EPROM, EEPROM, registers, a hard disk, a removable disk, tape, CD-ROM, DVD, floppy disk, Blu-ray disk, USB drive, or any other form of storage medium known in the art. The database 140 is a tangible storage medium for storing data and executable software instructions that are non-transitory during the time software instructions are stored therein. The database 140 may be secure and/or encrypted, or unsecure and/or unencrypted.

“Memory” and “database” are examples of computer-readable storage media, and should be interpreted as possibly being multiple memories or databases. The memory or database may for instance be multiple memories or databases local to the computer, and/or distributed amongst multiple computer systems or computing devices.

The controller 120 illustratively includes or has access to an AI engine, which may be implemented as software that provides artificial intelligence (e.g., inference of BFS, bolus duration and table feed speed) and applies machine-learning described herein. The AI engine, which provides a computational model described below, may reside in any of various components in addition to or other than the controller 120, such as the memory 130, the database 140, an external server, and/or a cloud, for example. When the AI engine is implemented in a cloud, such as at a data center, for example, the AI engine may be connected to the controller 120 via the internet using one or more wired and/or wireless connection(s). The AI engine may be connected to multiple different computers including the controller 120, so that the artificial intelligence and machine-learning described below in connection with various representative embodiments are performed centrally based on and for a relatively large set of medical facilities and corresponding subjects at different locations. Alternatively, the AI engine may implement the artificial intelligence and the machine-learning locally to the controller 120, such as at a single medical facility or in conjunction with a single imaging device 110.

The interface 160 may include a user and/or network interface for providing information and data output by the controller 120 and/or the memory 130 to the user and/or for receiving information and data input by the user. That is, the interface 160 enables the user to enter data and to control or manipulate aspects of the processes described herein, and also enables the controller 120 to indicate the effects of the user's control or manipulation. The interface 160 may include one or more of ports, disk drives, wireless antennas, or other types of receiver circuitry. The interface 160 may further connect one or more user interfaces, such as a mouse, a keyboard, a mouse, a trackball, a joystick, a microphone, a video camera, a touchpad, a touchscreen, voice or gesture recognition captured by a microphone or video camera, for example.

The display 150 may be a monitor such as a computer monitor, a television, a liquid crystal display (LCD), a light emitting diode (LED) display, a flat panel display, a solid-state display, or a cathode ray tube (CRT) display, or an electronic whiteboard, for example. The display 150 may also provide a graphical user interface (GUI) 155 for displaying and receiving information to and from the user.

As alluded to above, and as described in more detail below, in accordance with various representative embodiments, US imaging is used along a portion of the body to determine BFS prior to a medical imaging procedure, such as CTA imaging. The US methods and devices contemplated for ascertaining BFS are illustratively, but not limited to color Doppler imaging or 3D flow volumetry at specific locations/anatomical landmarks along the portion of the body being evaluated. In the description below, the blood flow is measured in a limb, specifically the legs prior to CTA imaging. It is emphasized that the application to a limb/legs is merely illustrative, and the present teachings are contemplated to evaluate the interference (e.g., stenosis) of normal blood flow in arteries and veins in other locations of the body.

Illustratively, the initial US imaging is used to identify main arteries and veins above (femoropopliteal) and below (infra-popliteal) the knee of a patient, and to identify, if possible, new collateral vessels. During the US sequence, cardiac physiological parameters (CPP) (e.g., heart rate, blood pressure) of the patient 105 are assessed, for use as described more fully below. A computational model is developed using deep-learning to infer BFS along all locations of the portion of the body (e.g., the leg) for a current patient (e.g., patient 105) by inputting only current parameters, including the US measurements from a plurality of specific locations on the portion of the body and cardiac physiological parameters of the current patient 105. Notably, in accordance with representative embodiments, the development of the computational model is part of the development of the system 100. This computational model is provided as a fully trained model as software to be executed by a processor of the computer system 115 as described more fully below.

The computational model is then used to infer current local BFS conditions immediately before CTA imaging is effected in the current patient 105. Specifically, the model receives as input the US-derived stenosis degree, local BFS information in peripheral vessels, and the CPPs; and outputs approximated BFS in lower limbs to aid with selection of imaging parameters including the speed of the table 106 upon which the patient 105 is moved into and out of the gantry doing the imaging, and the contrast injection protocols to provide a comparatively low (e.g., minimum necessary) bolus duration based on the inferred blood flow conditions and/or table feed speed. Notably, and as described below, the table feed speed can be determined over time for imaging devices (e.g., CT scanners) with variable-pitch enabled tables. Alternatively, the table feed speed can be determined to be constant over the duration of the scan.

FIG. 2 is a simplified flow diagram showing a system 200 for imaging blood flow, according to a representative embodiment. Various aspects and details of the system 200 and the attendant method are common to those described above, and may not be repeated in order to avoid obscuring the discussion of the present representative embodiment.

Initially, US scans are made of a patient using a suitable US imaging device 210, such as a color Doppler imaging or 3D flow volumetry imaging. Notably, the US imaging device 210 is typically operated separately from, and before the contrast imaging (e.g., CT). During the initial US imaging of a patient in a supine position, US images are gathered using the US imaging device 210 at specific locations 201, 202, 203, 204, 205 of the leg. Next, during the initial US imaging with the patient in a prone position, US images are gathered at specific locations 206, 207 of the leg. Generally, these specific locations 201˜207 are standard assessment points known to those of ordinary skill in the art. Notably, in certain representative embodiments, the specific locations 201˜207 are spaced at comparatively small distances apart (e.g., one location every 5 cm).

Next, at computational model 211, specified cardio physiological parameters (i.e. heart rate, blood pressure) are gathered for the patient. The US images from the specific locations 201˜207 and the CPPs are stored in a suitable database (e.g., database 140), which may include a known picture archiving and communication system (PACS) 209.

The input parameters comprising the BFS at the specific locations 201˜207 are then provided to the computational model 211, along with a current set of CPPs for the particular patient. The computational model 211 takes the input parameters including heart rate and blood pressure 212 to infer BFS's at all locations of the portion of the body (here, the legs) being examined. Notably, based on the model, varying degrees of BFS are determined by the model. For example, based on the output of the computational model 211, from the iliac artery 220, BFS in portions 221 is comparatively high, in portions 222 is comparatively low, and in portion 223 is between the BFS's in portions 221 and 223.

As described more fully below, the computational model 211 compiles ground truth data (GTD) from the measured BFS at the plurality of locations of an examined portion of a body, including each of the plurality of specific locations. The GTD are compiled in a study with selected participants. The computational model is then trained with the GTD, and the BFS is inferred throughout the portion of the body based US BFS measurements at a selected number of specific locations on the portion of the body to be imaged.

Notably, the GTD are used to train the computational model (i.e. artificial intelligence (AI) models based on neural and convolutional networks) and are generally not stored in the imaging system or US system of the representative embodiments. Rather, and as will become clearer as the present description continues, using measurements from a selected number of specific locations on the portion of the body to be imaged (sometimes referred to as key-point measurements), the (trained) computational model predicts flow speed throughout the limb.

The inferred BFS's are then provided to a controller (e.g., controller 120), which determines estimated ranges 230 for the table feed speed (variable or constant) for an imaging device 232 with a table 233, or a contrast medium injection duration using contrast injector 234, or both. As described more fully below, from these predicted BFS's, by way of lookup table or algorithm, a CM injection speed and duration, or table feed speed, or both, are determined and used to program the devices for the image scan.

Beneficially, an image (e.g., CT scan) of the portion of the patient is provided showing poor circulation, for example, in a region 236 of the leg.

As will be appreciated by one of ordinary skill in the art having the benefit of the present disclosure, the systems and methods of the present teachings provide an improvement in the function of an imaging system, such as the CT scanners described herein, and an improvement to the technical field of contrast-based medical imaging. For example, compared to known methods and systems, by determining the speed(s) of the table used in the scan, an overall reduction in the exposure of a patient to potentially harmful X-radiation is realized by the present teachings. Similarly, by determining the bolus duration based on the computational model, the overall dose (and consumption) of CM can be reduced compared to known methods. Notably, the benefits are illustrative, and other advancements in the field of medical imaging will become apparent to one of ordinary skill in the art having the benefit of the present disclosure. Moreover, the methods and systems of the various representative embodiments can only be implemented on a computer given the size and complexity of the task at hand, and cannot be practically be performed in the human mind.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

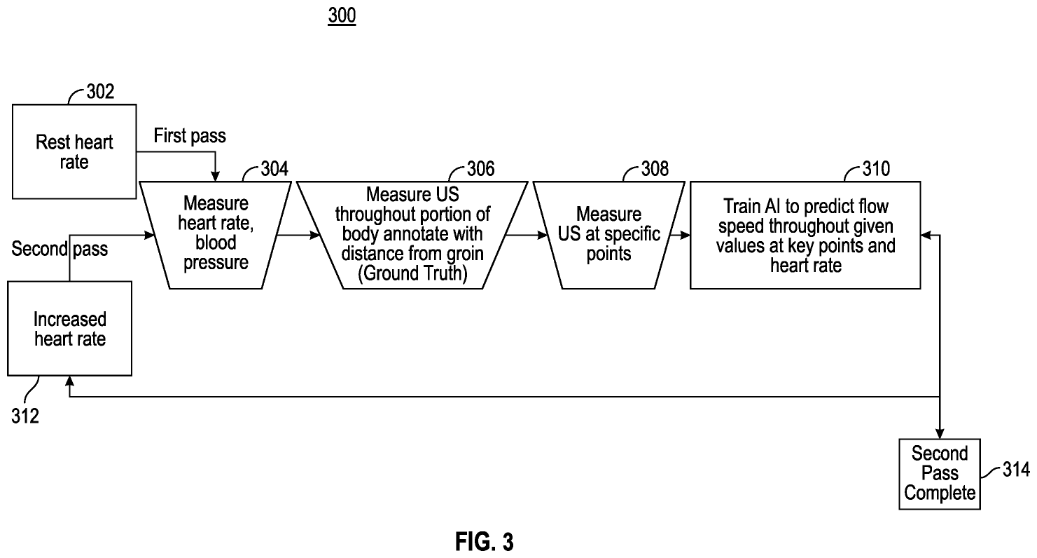

FIG. 3 is a simplified flow diagram 300 showing a method of training a computational model to predict BFS using the ground truth data, according to a representative embodiment. Various aspects and details of the system 200 and the attendant method are common to those described above, and may not be repeated in order to avoid obscuring the discussion of the present representative embodiment. Most notably, the method of training the computational model described in connection with FIG. 3 is contemplated for not only the method, but also the tangible, non-transitory computer readable medium that stores instructions, which when executed by a processor cause the processor to carry out the method of training the computational model.

As noted above, the method of FIG. 3, which includes compiling of the GTD from measured BFS at a plurality of locations of an examined portion of a body, including each of a plurality of specific locations, and the training of the computational model to predict BFS using the GTD, more generally be done by a processor or stored in a memory that are not part of the computer system 115. Rather, this compilation and storing of GTD and training of the computation model of the present teachings may be done using another processor and memory that are not necessarily part of the imaging system 100. In this case, after being trained, the computational model may be stored as executable instructions in memory 130, for example.

At 302, a resting heart rate for a patient is measured. At 304, the heart rate and blood pressure for the patient are measured and stored (e.g., in database 140) for a first pass of the method. As alluded to above, the CPPs comprise the heart rate and blood pressure, and when executing the computational model of various representative embodiments, are some of the input parameters to infer BFS, and determine table feed speed, and/or CM duration and injection rate. At 306, US imaging is applied to the entire portion of the body to be imaged by CT scan. As noted above in connection with the description of FIG. 2, in a representative embodiment, the portion of the body includes the legs.

At 306, actual US image data are gathered, and are annotated to reflect the distance of each data point on the leg from the groin. These data are saved and form GTD for the training of the computational model of the various representative embodiments. Notably, these GTD are gathered from a comparatively large number of patients (e.g., hundreds of people with circulation problems) by applying, for example, 3D flow volumetry US or applying color Doppler US. The patients assessed for compilation of GTD are generally referred to as “participants of the ground-truth compilation study.” In general, as for any GTD-compilation study, the condition of the participants of the ground-truth study (health status, physical characteristics, ethnicity, gender, etc.) are reflective of the patients whose properties (in this case throughout-limb BFS) are to be inferred.

At 308, US is applied at a selected number of specific locations on the portion of the body to be imaged. For example, with reference again to FIG. 2, US may be applied at specific locations 201˜207, which may be standard assessment points known to those of ordinary skill in the art. The data gathered at 308 are also from participants of the ground-truth compilation study, and as such, the data gathered at 308 are also GTD. Notably, as alluded to above, the BFS data gathered at 308 are also input parameters to the computational model that is used to infer BFS at in the portion of the body being scanned, which, in turn, is used to select the table feed speed, and/or bolus duration. Furthermore, the inclusion of 308 as a seemingly separate step is merely illustrative. Rather applying US to the selected number of specific locations can be completed in 306 when GTD are gathered over the entire portion of the body to be scanned.

At 310, training of the computational model (or AI model, or machine-learning algorithm) is carried out for the first pass. Training of the computational model generally happens during product development, and as noted above may not be done using computer system 115, but more generally may be completed using another memory and processor, with the trained computational model being stored in memory 130. The training of the computational model is based on ground-truth measurements on volunteering patients that comprise CPPs, a limited set of US BFS measurements at a plurality of specific (key-point) locations (example input), as well as BFS measurements at all locations in the limb(s) (example output) and follows the general steps described herein.

The training sequence of the computational model is carried out using one of a number of known machine-learning techniques known to those of ordinary skill in the art of AI and mathematical models. In machine-learning, an algorithmic model “learns” how to transform its input data into meaningful output. During the learning sequence, the computational model is shown known examples of inputs and their correct or desired output. These examples are called ground truth, and in accordance with the present teachings comprise the GTD gathered at 306 and 308. During the learning sequence, the computational model adjusts its inner parameters given the input parameters of the examples, and produces the corresponding meaningful output or so-called target. The adjustment process is guided by instructions on how to measure the distance between the currently produced output and the desired output. These instructions are called the objective function.

In deep learning, which is a subfield of machine-learning, the inner parameters inside the computational model are organized into successively connected layers, where each layer produces increasingly meaningful output as input to the next layer, until the last layer which produces the final output.

Deep learning layers are typically implemented as so-called neural networks, that is, layers are comprised of a set of nodes, each representing an output value and a prescription on how to compute this output value from the set of output values of the previous layer's nodes. The prescription being a weighted sum of transformed output values of the previous layer's nodes, each node only needs to store the weights. The transformation function is the same for all nodes in a layer and is also called activation function. There are a limited number of activation functions that are used today. A particular way to set which previous layer's nodes provide input to a next layer's node is convolution. Networks based on this way are called convolutional networks.

Thus, in the learning phase or so-called training, for each example, the output of the final layer is computed. Outputs for all examples are compared with the desired outputs by way of the objective function. The output of the objective function, the so-called loss, is used as a feedback signal to adjust the weights such that the loss is reduced. The adjustment, i.e. which weights to change and by how much, is computed by the central algorithm of deep learning, so-called backpropagation, which is based on the fact that the weighted sums that connect the layer nodes are functions that have simple derivatives. The adjustment is iterated until the loss reaches a prescribed threshold or no longer changes significantly.

A deep-learning network thus can be stored (e.g., in database 140 or memory 130) as a topology that describes the layers and activation functions and a (large) set of weights (simply values). A trained network is the same, only the weights are now fixed to particular values. Once the network is trained, it is put to use, that is, to predict output for new input for which the desired output is unknown.

In the present teaching, the computational model is stored as instructions to provide the machine-learning algorithm, such as a convolutional neural-network deep learning algorithm. When executed by a processor, the machine-learning algorithm (the computational model) of the representative embodiments is used to infer BFS at all locations in the portion of the body to be CT scanned (e.g., limb(s)) from measurements at specific locations (e.g., specific locations 201˜207) of the portion of the body. This limited set of input parameters may include, but are not limited to, CPPs, US BFS data from the specific locations (e.g., specific locations 201˜207) of the portion of the body, low speed measurements at key-point locations, plus heart rate and blood pressure.

Upon completion of the first pass, at 312 the heart rate is increased either through physical exertion or pharmacological stimulation, and the steps 304-310 are repeated. After gathering GTD through the second pass, at 314, the training method is completed for this round of training.

As alluded to above in connection with the description of FIG. 2 and as described more fully below, US BFS measurements are taken shortly before the image scan (CT) is done. With the noted input parameters, the computational model predicts or infers BFS at a plurality (e.g., along the entire length) of portions of the body to be scanned (e.g., in the limb(s)). Moreover, a method is provided for determining table feed speed and/or contrast injector settings to optimize the scan, and to reduce the radiation exposure time and bolus duration compared to known systems and methods. As described below, the method of determining table feed speed and/or contrast injector settings, while generally not a machine-learning algorithm, is stored as executable instructions, which when executed by a processor of controller 120, computes the table feed speed and/or contrast injector settings for the patient and scanning system being used. As will be readily appreciated, the faster the blood flows, the faster the table needs to travel and the longer the CM injection needs to be.

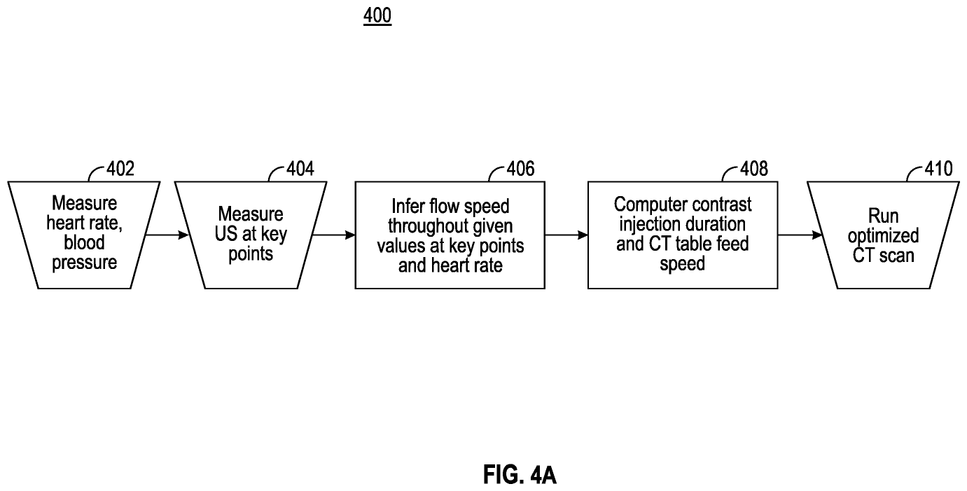

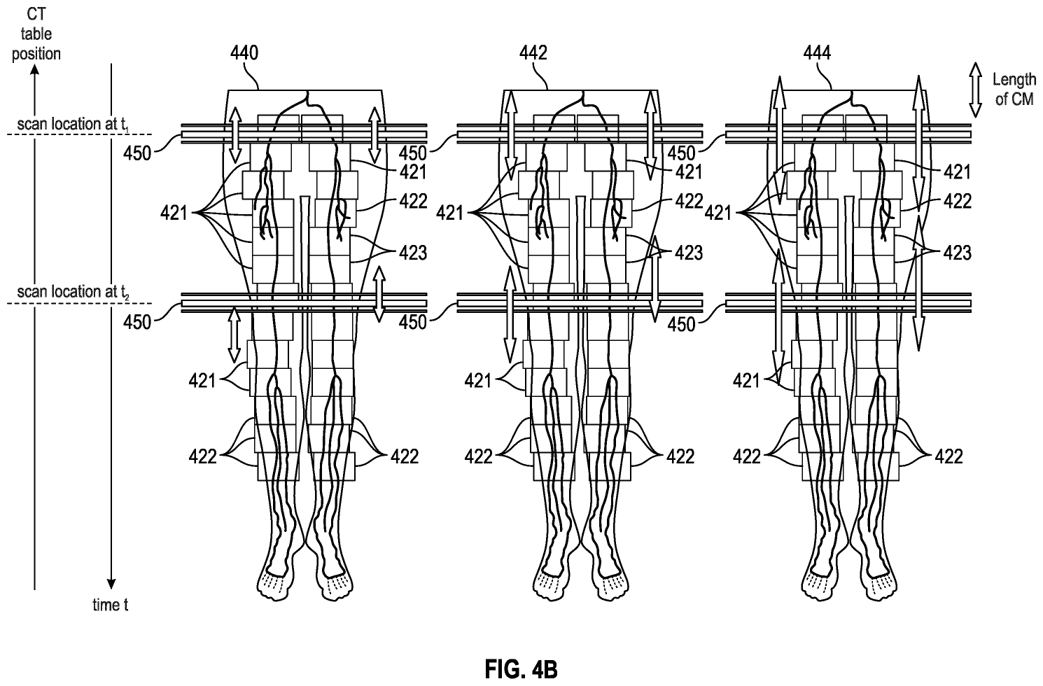

FIG. 4A is a simplified flow diagram showing a method 400 of inferring BFSs throughout a portion of the body based on the computational model. The flow diagram also includes the determination of contrast injection duration and table feed speed noted above. Various aspects and details of the flow diagram and the attendant method 400 are common to those described above, and may not be repeated in order to avoid obscuring the discussion of the present representative embodiment. Most notably, the method 400 is contemplated for not only the method, but also for storing in a tangible, non-transitory computer readable medium that stores instructions, which when executed by a processor, cause the processor to carry out the method of inferring (predicting) BFS throughout the portion of the body to be scanned. Moreover, and as described more fully below, the inferring or predicting of BFS throughout the portion of the body is based on key-point measurements. From that inferred BFS through the entire portion of the body, and factoring the detector width of the image scanner, and the injection rate of the CM injector being used, table feed speed and/or CM injection duration are determined from a look-up table or functionally or algorithmically computed (i.e., stored as instructions to be executed by a processor). Notably, the executable instructions for inferring the BFS, and the executable instructions or look-up table to determine the table feed speed and/or CM injection duration can stored separately or together in persistent storage.

The method starts at 402 where CPP (e.g., heart rate, blood pressure) are measured. Notably, this step may be carried out manually, or using known electronic devices that are part of the imaging system 100, for example.

At 404 US BFS data are gathered by applying US (e.g., three-dimensional (3D) flow volumetry US or color Doppler US) at the specific locations (e.g., specific locations 201˜207) of the portion of the body. Notably, these US measurements are carried out on the current patient typically twice and shortly before the scan (CT) are done on the current patient.

At 406 the input parameters including the measured heart rate, blood pressure, and US BFS data from 402 and 404 are input to the computational model trained in accordance with representative embodiments described above. The computational model, which, as noted above, is stored as executable instructions in a tangible, non-transitory computer readable medium, infers (or predicts) the BFS throughout the portion of the body. Specifically, a processor in the controller 120 executes the computational model to predict the BFS at a plurality of locations of the portion of the body (e.g., a leg) to be scanned.

At 408, the inferred BFS data are used to determine the contrast injection duration, or table feed speed, or both, for the current patient. In accordance with a representative embodiment, the contrast injection duration and the table feed speed (constant or variable) are stored in a look-up table for various injector volume speeds, table feed speeds, and detector width used in the scan of the current patient. Specifically, for a given set of BFS data gathered for the current patient, the lookup table provides table feed speed, or injector volume speed, or both, for a particular detector width used in the scan.

Alternatively, the table feed speed or CM injection rate, or both, for a particular scan can be determined functionally or algorithmically by executable instructions, which when executed by a processor determine the table feed speed and/or CM injection rate.

As noted above, the table feed speed is matched to a determined bolus duration for the current patient. As such, the table feed speed is selected to match CM injection rate during the scan. The matched table feed speed or CM injection rate, or both, selected from the look-up table, or determined functionally or algorithmically, beneficially minimize both the radiation exposure time and CM volume compared to known techniques and systems used on image scanning, such as the illustrative CT scans of the representative embodiments.

At 410, an optimized CT scan is run using the determined table feed speed and/or bolus duration of the CM. Again, while a CT scan is used to describe various illustrative embodiments, the present teachings are contemplated for use in other image scanning methods and systems that use CM injection, or apply radiation to effect the scan, or both. Beneficially, by the present teachings, the volume of the CM injected, or the duration of exposure to radiation by a patient, or both, are reduced or minimized compared to other known systems and methods.

FIG. 4B shows contrast media injection levels in limbs useful in understanding the benefits of various aspects of the representative embodiments. Various aspects and details of the system of the description of FIG. 4B are common to those described above, and may not be repeated in order to avoid obscuring the discussion of the present representative embodiment.

FIG. 4B scans 440, 442 and 444 show varying degrees of CM injection duration (time) for a patient, which is commonly referred to as the length of the CM. Notably, based on the output of the computational model according to a representative embodiment, BFS in portions 421 is comparatively high, in portions 422 is comparatively low, and in portion 423 is between the BFS's in portions 421 and 422.

As is known, the faster the flow rate of CM through the blood passing through location where a CT scan is carried out, the length of the CM, which is also referred to as the “train” of the CM, must be greater. As described herein, it is beneficial to provide a comparatively short length of the CM so that the CT scanner is able to acquire the X-ray projection data with good contrast. Beneficially, the shortest length of the CM should be provided for the CT scanner to acquire the X-ray projection data with good contrast. This in turn, reduces the exposure of the patient to the CM media. CT scan rates are generally fast enough to properly track blood flow at typical BFS. However, because the BFS varies throughout the portion of the body being scanned (e.g., BFS in portions 421, 422, 423 differ as predicted by the computational model), and the table feed speed is not always variable, the CM injection needs to be long enough so the CT scanner table can temporarily slow down in regions where the blood flow is comparatively slower, or speed up in regions where the blood flow is comparatively faster, and still remain well within the region of good contrast. Accordingly, the injection duration is comparatively longer relative to an inferred slow blood speed, and comparatively shorter relative to an inferred fast blood speed. For tables with variable feed speed, by the present teachings, the table feed speed can be varied based on the computational model to match the expected location of the contrast media in a specific leg. Depending on the BFS in the other leg, the CM duration can be adjusted by the computational model accordingly to ensure presence of CM at the relevant portions of both legs that coincide with the detector position at different times. If the table speed is constant, then the speed is set initially at the desired level based on one of the BFSs in the diseased leg (either lowest detected BFS or highest or some in-between or average value), and the CM duration is adjusted by the computational model as in the earlier case.

In accordance with representative embodiments, based on the inferred BFS's from the computational model, the contrast medium injection duration and the feed speed of the table prior to the CT scan can be determined.

With reference to FIG. 4B, a time t₁, contrast has advanced evenly in both legs. Due to disease, blood and CM flow are comparatively slowed in the upper left leg. As such, at some later t₂, the CM has not advanced as far in the left leg as in the right leg. In scan 442, there is no contrast in right leg; in scan 44444, the amount of CM in the legs is too great; and in scan 442, the proper contrast is achieved with the a comparatively small amount of CM in the legs. Stated somewhat differently, scan 442 shows a sufficiently long contrast section compared to the scan 440; and scan 444 shows a contrast section that is unnecessarily long. By the present teachings, by proper selection of table feed speed and/or CM injection duration, poor scans such as scan 440 are avoided, whereas injecting unnecessary amounts of CM such as scan 444 are also avoided. Among other benefits, by reducing the number of poor scans (e.g., 440), and avoiding injecting more CM into a patient that is needed for a scan (e.g., scan 444), patient exposure to radiation, and too much CM, respectively, are avoided.

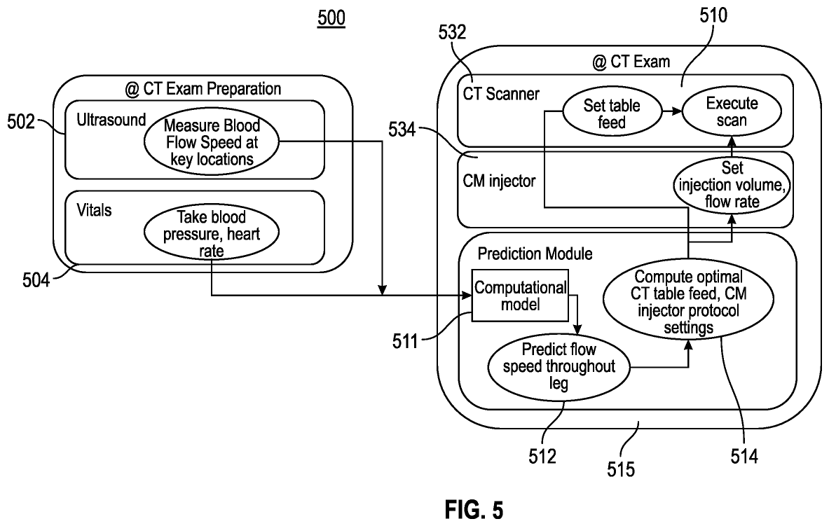

FIG. 5 is a simplified block diagram of a system 500 for conducting an imaging scan, according to a representative embodiment. Various aspects and details of the system 500 are common to those described above, and may not be repeated in order to avoid obscuring the discussion of the present representative embodiment.

The system 500 comprises an US device 502 adapted to apply US to specific locations of a body of a current patient. As described above, the US device 502 measures BFS at the specific locations at the specific locations.

The system 500 also comprises a vital signs measurement device 504 that is adapted to measure the heart rate and blood pressure of the current patient as described above.

The BFS measurements and CPP measurements are then provided to a computational model 511 of a computer system 515, which is used for controlling imaging of a region of interest of a patient. The computer system 515 includes various components described above in connection with computer system 115, but not shown in FIG. 5, including the controller 120, the memory 130, and the database 140.

A computational model 511 is stored as instructions executable by a processor to infer BFS's throughout the portion of the body based on the computational model. Specifically, the computational model 511 receives the BFS data a CPP data from the US device 502 and vital signs measurement device 504 and at 512 infers the BFS at a plurality of locations of the portion of the body to be scanned, such as described above.

The BFS predicted at 512 is input at 514 to determine the table feed speed and/or the CM injector settings as described above.

The table feed speed and/or CM injector settings are provided to an imaging device 510. Specifically, the CM injector flow rate of a CM injector 534 is set, and/or the table feed speed of a CT scanner 532 is set.

Finally, and as noted above, according to a representative embodiment, the computational model 511 may be stored in a memory and executed by a processor that is not part of the computer system 515, but rather is connected to the imaging device 515 through an external link (not shown). Just by way of illustration, the computational model may be stored as executable instructions in a memory, and executed by a server that is remote from the imaging device (e.g., a CT scanner). When executed by the processor in the remote server, the instructions cause the processor to compile ground truth data (GTD) from measured BFS at a plurality of locations of an examined portion of a body, including each of a plurality of specific locations; train a computational model 511 to predict BFS using the ground truth data; and infer BFS's throughout the portion of the body based on the computational model 511. Moreover, the remote server may be configured to determine a contrast medium injection duration, or a speed of a table, or both, prior to an image scan based on the inferred blood flow speeds. The determined table feed speed and/or CM injector settings are then provided by the server to an imaging device 510. Specifically, the CM injector flow rate of a CM injector 534 is provided, and/or the table feed speed of a CT scanner 532 is provided for the CT scanner 532 to effect an imaging run.

Thereafter, the scan, which in this illustrative embodiment is a CT scan, is carried out by the imaging device 111.

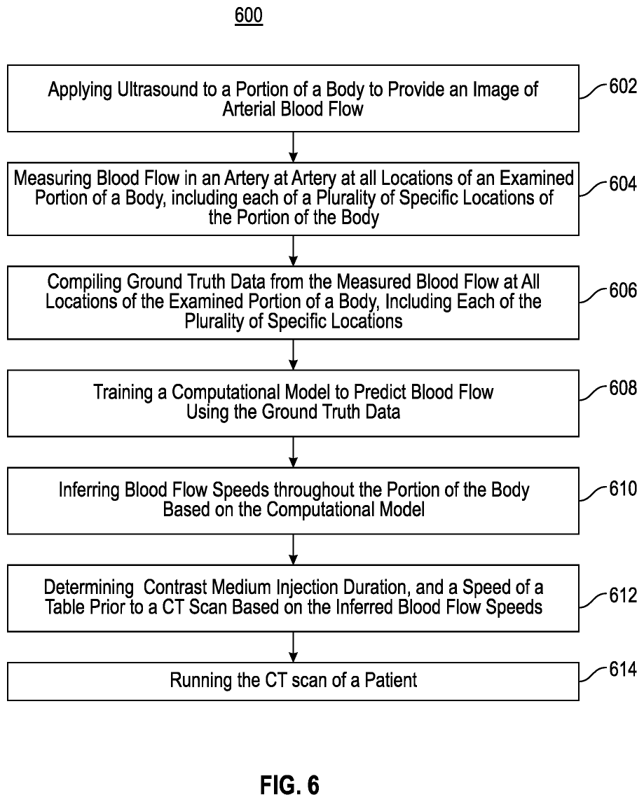

FIG. 6 is a simplified flow diagram showing a method 600 of imaging a portion of a body including training a computational model, according to a representative embodiment. Various aspects of the method and details of the flow diagram and the attendant method 400 are common to those described above, and may not be repeated in order to avoid obscuring the discussion of the present representative embodiment. Most notably, the method 600 is contemplated for not only the method, but also for storing in a tangible, non-transitory computer readable medium that stores instructions, which when executed by a processor, cause the processor to carry out the method 600. Finally, while the method 600 is contemplated for use in image-based angiography, the method 600 may be used in other systems where minimizing CM exposure or radiation exposure of a patient is desired.

At 602, the method 600 comprises applying US to a portion of a body to provide an image of arterial blood flow.

At 604, the method 600 measuring BFS in an artery at a plurality of locations of an examined portion of a body, including a plurality of specific locations of the body.

At 606, the method 600 comprises compiling ground truth data from the measured BFS at the plurality of locations of an examined portion of a body, including each of the plurality of specific locations.

At 608, the method 600 comprises training a computational model to predict BFS using the ground truth data.

At 610, the method 600 comprises inferring BFS's throughout the portion of the body based on the computational model.

At 612, the method 600 comprises determining contrast medium injection duration, and a speed of a table prior to an image scan based on the inferred BFS's.

At 614, the method 600 comprises running an image scan of a patient.

Although methods, systems and components for determining CM injection rates and table feed speed useful in image scanning to reduce the exposure of a patient to radiation and to limit the volume of CM injection needed for an imaging scan has been described with reference to several exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Although developing adaptable predictive analytics has been described with reference to particular means, materials and embodiments, developing adaptable predictive analytics is not intended to be limited to the particulars disclosed; rather developing adaptable predictive analytics extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

Although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method performing an image-based angiography, the method comprising:
applying ultrasound to a portion of a body to provide an image of arterial blood flow;
measuring blood flow speed in an artery at a plurality of locations of an examined portion of a body, including a plurality of specific locations of the body;
compiling ground truth data from the measured blood flow speed at the plurality of locations of an examined portion of a body, including each of the plurality of specific locations;
predicting using a computational model a predicted blood flow speed using the ground truth data;
inferring blood flow speeds throughout the portion of the body based on the computational model; and
determining contrast medium injection duration, or a speed of a table, or both, prior to an image scan based on the inferred blood flow speeds.

2. The method of claim 1, further comprising, prior to the measuring blood flow speed, assessing a heart rate and a blood pressure of a patient.

3. The method of claim 2, further comprising, after the compiling ground truth data, changing the heart rate of the patient, and repeating the applying the ultrasound, the measuring blood flow speeds at the plurality of specific locations, and the compiling ground truth data.

4. The method of claim 1, wherein the compiling the ground truth data further comprises determining ultrasound-derived stenosis degree and a speed of local blood flow.

5. The method of claim 1, wherein the determining the contrast medium injection duration further comprises determining a contrast injection protocol to provide a lowest bolus duration for the inferred blood flow speed and the speed of the table.

6. The method of claim 1, wherein the specific locations comprise locations of common stenosis.

7. The method of claim 1, wherein the predicting with the computational model further comprises identifying locations of stenosis based on the applied ultrasound and determining the specific locations from the identified locations.

8. The method of claim 1, wherein the applying ultrasound further comprises applying three-dimensional flow volumetry ultrasound or applying color Doppler ultrasound.

9. The method of claim 1, wherein the determining the speed of the table further comprises determining from the specific locations where a relative speed is increased based on the blood flow speed, and determining from the specific locations where a relative speed is decreased based on the blood flow speed.

10. The method of claim 1, wherein the determining the speed of the table further comprises determining a constant speed based on the blood flow speed at the specific locations.

11. A system for medical imaging, comprising:
a computer tomography device comprising a table;
a processor;
a memory that stores instructions, which when executed by the processor, cause the processor to:
compile ground truth data from measured blood flow speed at a plurality of locations of an examined portion of a body, including each of a plurality of specific locations;
infer blood flow speeds throughout the examined portion of the body based on a computational model;
determine a contrast medium injection duration, or a speed of a table, or both, prior to a CT scan based on the inferred blood flow speeds; and
provide the determined contrast medium injection duration, or the speed of the table, or both.

12. The system of claim 11, wherein the processor is further configured to train the computational model to predict blood flow speed using the ground truth data;

13. The system of claim 11, wherein the instructions, when executed by the processor, further cause the processor to train the computational model to determine ultrasound-derived stenosis degree and a speed of local blood flow.

14. The system of claim 11, wherein the instructions, when executed by the processor to determine the contrast medium injection duration further cause the processor to determine a contrast injection protocol to provide a lowest bolus duration for the inferred blood flow speed and the speed of the table.

15. The system of claim 11, wherein the plurality of specific locations comprises locations of common stenosis.

16. The system of claim 12, wherein the instructions, when executed by the processor to train the computational model, further cause the processor to identify locations of stenosis based on an ultrasound scan, and to determine the plurality of specific locations from the identified locations.

17. A tangible, non-transitory computer readable medium that stores instructions, which when executed by a processor, cause the processor to:
compile ground truth data from measured blood flow speed at a plurality of locations of an examined portion of a body, including each of a plurality of specific locations;
predict with a computational model a predicted blood flow speed using the ground truth data;
infer blood flow speeds throughout the portion of the body based on the computational model;
determine a contrast medium injection duration, or a speed of a table, or both, prior to a CT scan based on the inferred blood flow speeds; and
provide the determined contrast medium injection duration, or the speed of the table, or both.

18. The tangible, non-transitory computer readable medium of claim 17, wherein the instructions, when executed by the processor, further cause the computational model to determine ultrasound-derived stenosis degree and a speed of local blood flow.

19. The tangible, non-transitory computer readable medium of claim 17, wherein the instructions, when executed by the processor to determine the contrast medium injection duration further cause the processor to determine a contrast injection protocol to provide a lowest bolus duration for the inferred blood flow speed and the speed of the table.

20. The tangible, non-transitory computer readable medium of claim 17, wherein the plurality of specific locations comprises locations of common stenosis.

21. The tangible, non-transitory computer readable medium of claim 17, wherein the instructions, when executed by the processor, further cause the processor to identify locations of stenosis based on an ultrasound scan, and determine the plurality of specific locations from the identified locations.
